(19) United States
(12) Patent Application Publication
(10) Pub. No.: US 2009/0171166 A1
(43) Pub. Date: Jul. 2, 2009

(54) OXIMETER WITH LOCATION AWARENESS

(75) Inventors: Scott Amundson, Boulder, CO (US); Corydon A. Hinton, Oakland, CA (US); Li Li, Milpitas, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/340,978

(22) Filed: Dec. 22, 2008

Related U.S. Application Data
(60) Provisional application No. 61/009,705, filed on Dec. 31, 2007.

(51) Int. Cl. A61B 5/02; A61B 5/00

(52) U.S. Cl. 600/301; 600/424

(57) ABSTRACT

According to embodiments, systems and methods are provided for determining the location of a medical device. A system may include a plurality of wireless communication devices configured to receive signals from a medical device and a location server configured to receive information from the plurality of wireless communication devices and determine the location of the device within a specified range. A method of operation may include receiving a plurality of signals corresponding to a medical device from a plurality of wireless communication devices and determining the location of the medical device from the plurality of signals. A sensor and monitor configured to transmit location information to one or more wireless communication devices are also provided.

### RELATED APPLICATION

This application claims priority from U.S. Provisional Application No. 61/009,705, filed, Dec. 31, 2007, which is hereby incorporated by reference herein in its entirety.

### BACKGROUND

The present disclosure relates generally to medical devices and, more particularly, to locating medical sensors, patient monitors, and other medical devices.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Pulse oximeters and other medical devices are typically mounted on stands that are positioned around a patient's bed or around an operating room table. When a caregiver desires to command the medical device (e.g., program, configure, and so-forth), the caregiver manipulates controls or pushes buttons on the monitoring device itself. The monitoring device typically provides results or responses to commands on a liquid crystal diode (“LCD”) screen mounted in an externally visible position within the medical device. Patient data, alerts, and other information may be displayed on the monitor directly, or may be transmitted over a wired link to a central computer monitored by caregivers.

As these medical devices become smaller and more portable, locating and securing these medical devices may become more challenging. For example, in the case of a wearable sensor worn by a patient, the patient may wander to a different area or attempt to leave a hospital or other medical facility. Further, even if the devices are not secured to a patient, the size of the devices may also result in misplacement. Additionally, as patient monitors also decrease in size, the monitor may be misplaced in the wrong room, mistakenly put into storage, etc.

Not only is replacement of these medical devices an undesirable expense, theft is also a concern. As the medical devices become smaller and more portable, they are more easily hidden and removed from a hospital or other medical environment. The size of the devices and the desire for increased portability may limit the use of technology to prevent the devices from misplacement or theft.

### SUMMARY

Certain aspects commensurate in scope with the disclosure are set forth below. It should be understood that these aspects are presented merely to provide the reader with a brief summary of certain forms of the disclosure might take and that these aspects are not intended to limit the scope of the disclosure. Indeed, the disclosure may encompass a variety of aspects that may not be set forth below.

In one embodiment, a medical device is provided that includes a sensor configured to monitor a physiological parameter, wherein the sensor is configured to transmit location information to one or more wireless communication devices.

In another embodiment, a medical device is provided that includes a patient monitor configured to monitor a physiological parameter, wherein the monitor is configured to transmit location information to one or more wireless communication devices.

A method of operation is provided that includes receiving a plurality of signals corresponding to a medical device from a plurality of wireless communication devices and determining the location of the medical device from the plurality of signals.

In another embodiment, a system is provided that includes a plurality of wireless communication devices configured to receive signals from a medical device and a location server configured to receive information from the plurality of wireless communication devices and determine the location of the device within a specified range.

### BRIEF DESCRIPTION OF THE DRAWINGS

Advantages of the disclosure may become apparent upon reading the following detailed description and upon reference to the drawings in which:

FIG. 1 depicts a locating system in use with a wearable system in accordance with an embodiment;

FIG. 2 depicts a locating system in use with a patient monitor in accordance with an embodiment;

FIG. 3 illustrates a multiple-area locating system in accordance with an embodiment;

FIG. 4 is a block diagram of a sensor and monitor in accordance with an embodiment; and

FIG. 5 is a flowchart of a process for locating a medical device in accordance with an embodiment.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

It may be desirable to provide a system for locating or tracking the location of a medical device, such as wearable sensor or monitor. The location may be stored over a period of time to further aid in tracking the device. Further, alerts or other notifications may be sent to devices on a hospital network based on the location of the device.

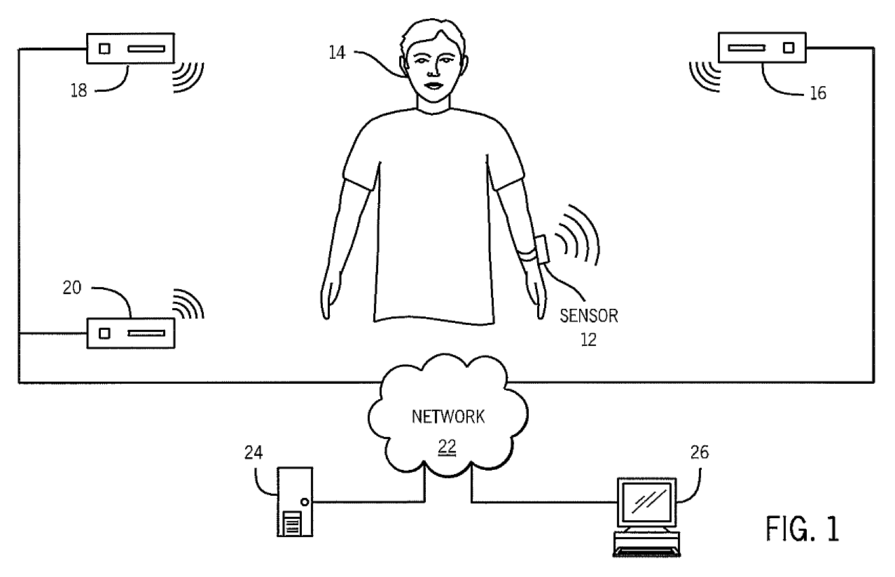

Turning now to the figures, FIG. 1 depicts a locating system 10 configured to locate a wearable medical sensor 12 worn by a patient 14 in accordance with an embodiment of the present disclosure. The locating system 10 may include a plurality of access points, such as a first access point 16, a second access point 18, and a third access point 20, the operation of which will be described further below. Each access point may be wirelessly or physically connected to a network 22. Other devices on the network include a location server 24 and a remote computer 26.

In an embodiment, the sensor 12 may be any sensor configured to monitor a physiological parameter and may be connected to a body part (e.g., finger, forehead, toe, or earlobe) of the patient 14. The sensor 12 may be configured to be clipped onto a finger or earlobe or may be configured to be secured with tape or another static mounting technique. For example, as a pulse oximetry sensor, the sensor 12 may clip onto a patient or user's finger and may be configured to emit signals or waves into the patient's or user's tissue and detect these signals or waves after dispersion and/or reflection by the tissue. More specifically, the sensor 12 may be configured to emit light from two or more light emitting diodes (“LEDs”) into pulsatile tissue (e.g., finger, forehead, toe, or earlobe) and then detect the transmitted light with a light detector (e.g., a photodiode or photo-detector) after the light has passed through the pulsatile tissue. In other embodiments, the sensor may be a reflectance-type pulse oximetry sensor, an electrocardiogram (EKG), a blood sugar (glucose) sensor, a blood pressure sensor, a temperature sensor, or any other sensor configured to monitor a physiological parameter. The sensor 14 may also include other items, such as a battery to provide power.

To communicate with access points 16, 18, and 20, the sensor 12 may include an active transmitting device. For example, in one embodiment, the active transmitting device may be an active radio frequency identification (RFID) device. As opposed to a passive device, the active RFID may periodically or continuously transmit information to any of the access points 16, 18, or 20. In this manner, the location of the sensor 12 may be determined via triangulation from the data received by the access points 16, 18, and 20.

The access points 16, 18, and 20 may provide, extend and/or use any type of wireless networking technology. For example, in one embodiment the access points 16, 18, and 20 may provide or use Wi-Fi (IEEE 802.11) networking standards. In other embodiments, the access points 16, 18, and 20 may provide or use WiMax, or any other suitable wireless networking technology. The access points 16, 18, and 20 may connect to a network 22, such as a local area network (LAN) or a wide area network (WAN). The network 22 may encompass a network for the entire hospital, or other medical facility where the patient 14 is located and the sensor 12 is being used. Thus, the access points 16, 18, and 20 may extend the range or accessibility of such a network.

Other devices may be included on the network 22, and they may be a part of the locating system 10 or may receive information related to the location of the sensor 12 and the patient 14. For example, a location server 24 may be physically or wirelessly connected to the network 22. The location server 24 may perform a number of functions as a part of the locating system 10. One of the primary functions of the location server 24 may be to analyze and process data received from the access points 16, 18, and 20. Thus, the location server 24 may perform the triangulation calculations necessary to determine the location of the sensor 12. Additionally, the location server 24 may store the location of the sensor 12 as determined from the data received from the access points 16, 18, and 20 in the triangulation. In one embodiment, the location server 24 may continuously receive data from the access points 16, 18, and 20, as the sensor's built-in active REID may continuously send data. In other embodiments, the sensor's built-in active RFID may periodically send data, such as every 5 minutes, 10 minutes, 15 minutes, 20 minutes, etc. In one embodiment, the locating system 10 may be a locating system provided by AeroScout, Inc of Redwood City, Calif.

Another device on the network 22 may be a remote computer 26 that may receive data from the locating system 10, or allow access to the data from locating system 10. In one embodiment, the remote computer 26 may be a monitoring computer located at a central desk or caregiver station in the hospital or other medical environment. In this embodiment, if the location server determines that the location of the sensor 12, and thus possibly the patient 14, is abnormal, then the location server 24 may send an alert to the remote computer 26. The remote computer 26 may respond to the alert by providing an audio and/or visual notification. In this manner, a caregiver, such as a nurse or doctor, located at the station where the remote computer 26 is located may receive a notification if the sensor 12 is outside the expected location. Such a notification may be critical if the sensor 12 may be outside the expected location, if the patient 14 is attempting to leave the hospital or medical facility, if the sensor has been stolen, misplaced, or any other undesirable reason.

In another embodiment, the remote computer 26 may be a workstation or a personal computer for a caregiver that allows access to the data generated by the location server 24. If the caregiver wishes to review or track the location of the sensor 12, the caregiver may use the remote computer 26 to pull up the stored data on the location server 24. Thus, a caregiver can determine the current location of the sensor 12, and the patient 14, and can also determine how much the patient 14 is moving or the areas they are moving through.

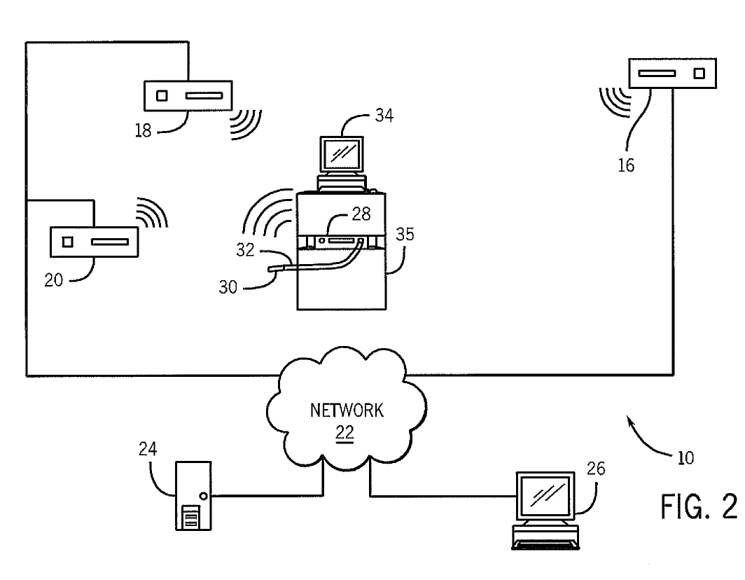

Turning now to FIG. 2, an embodiment of the locating system 10 is shown in use with a patient monitor 28. The monitor 28 may include a sensor 30, such as a pulse oximeter sensor or other sensor configured to monitor a physiological parameter of a patient. In the embodiment illustrated in FIG. 2, the monitor 28 and the sensor 30 may be initially located in a patient's room. The sensor 30 may be coupled to the monitor 28 via a sensor cable 32 to allow for communication between the sensor 28 and the monitor 30. In other embodiments, the sensor 28 may communicate with the monitor 30 via wireless technology such as radio, infrared, or optical signals. The monitor 28 may be housed in a cabinet 34, so as to allow movement around the patient's room, to another room, etc. The monitor 28 may also be connected to a display 34, which may display additional information received from the monitor 28.

In this embodiment, the monitor 28 may send and receive data from the sensor 30. In one embodiment, the monitor 28 may be a suitable pulse oximeter, such as those available from Nellcor Puritan Bennett Inc. In other embodiments, the patient monitor 28 may be a multi-purpose monitor suitable for performing pulse oximetry and/or measurement of any other physiological and/or biochemical parameter, using data acquired via the sensor 30. Accordingly, the sensor 30 may be a pulse oximeter sensor or any other sensor suitable for measuring a physiological parameter. Use of the monitor 204 may allow for use of a wider range of sensors, as opposed to the wearable sensors described above.

The monitor 28 may be moved via the cabinet 34, or, it may be removed from the cabinet 34 and taken to another location. In either case, it may be desirable to know or identify the location of the monitor 28. For example, the monitor 28 may be needed for use with a patient or may be needed for maintenance, updating, etc. To facilitate operation with the locating system 10, the monitor 28 may include an active transmitting device. For example, in one embodiment, the active transmitting device may be an active radio frequency identification (RFID) device. As opposed to a passive device, the active RFID may periodically or continuously transmit information to any of the access points 16, 18, or 20. Additionally, the size of the monitor 28 may allow for alternative or additional active transmitting devices, such as a Wi-Fi network interface or other wireless networking interface.

As described above, the location of the monitor 28 may be determined via triangulation from the data received by the access points 16, 18, and 20. Additionally, the location server may store the current location of the monitor 28 as well as a history of past locations, and it may provide alerts or notifications based on the location of the monitor as described above with regard to the sensor. Similarly, the remote computer 26 may allow retrieval and review of the location data and may aid in locating the monitor 28 when the monitor 28 is needed.

Further, the active transmitting capability of the monitor 28 in combination with the access points 16, 18, and 20 and the locating system 10 may provide for automatic registering of the monitor 28. For example, if the monitor 28 is a new monitor or is a reactivated old monitor, the monitor 28 may automatically register itself with the location server 24. The location server 24 may receive a signal from the new or reactivated monitor 28 and, upon comparison to a database of stored devices, recognize that the new or reactivated monitor 28 has not been used before. Upon recognition of a new or reactivated device, the location server 24 may be directly or indirectly (via another server such as remote computer 28) automatically add the device to a database, thus updating inventory or other records. Alternatively, the registration process may use any combination of manual and automatic actions. For example, instead of automatically adding a device to an inventory or other database, the location server 24 can send a notification to remote computer to notify a caregiver or administrator that a device needs to be added to a database.

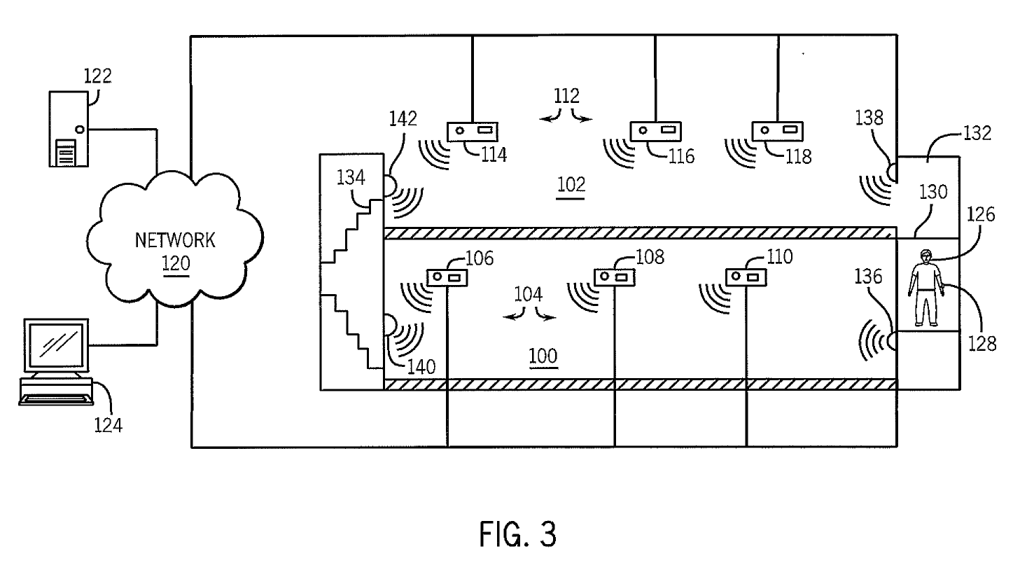

In other embodiments, it may be desirable to use a locating system to cover multiple floors or larger areas of a hospital or other medical facility. FIG. 3 depicts multiple locating systems on a first floor 100 and a second for 102 of a hospital or other medical facility in accordance with an embodiment of the present disclosure. A first locating system 104 that includes access points 106, 108, 110 may operate primarily on the first floor 100. A second locating system 112 that includes access points 114, 116, and 118 may operate primarily on the second floor 102. Each set of access points may be connected to a network 120. As discussed above, the first locating system 104 may use the access points 106, 108, and 110 to locate a device on the floor 100 via triangulation. Similarly, the second locating system 112 may use access points 114, 116, and 118 to locate medical devices on the second floor 102. The access point and locating systems 104 and 112 may operate according to the techniques described above with regard to wireless connectivity and so forth.

Included on the network 120 may be a location server 122 and a remote computer 124. The location server 122 may provide all the functions discussed above with respect to locating a medical device, storing the locations of a medical device, alerting and/or notifying a caregiver, and registering devices. Similarly, the remote computer 124 may provide the functions described above with respect to accessing and/or browsing the information stored on the location server 122.

However, a medical device located on the first floor 100 may not necessarily remain on the first floor 100, as it may be inadvertently or undesirably moved to another floor, such as the second floor 102. For example, a patient 126 wearing a wearable sensor 128 may move between the first floor 100 and the second floor 102 via an elevator 130 in an elevator shaft 132. Alternatively, the patient 126 may move between floors via a stairwell 134. In either case, it may be undesirable for the patient 126 or the sensor 128 to move from the floor in which the patient 126 or the sensor 128 is initially placed. Even though this embodiment is described with reference to a wearable sensor 128, it is also applicable to any other portable medical device, such as patient monitors, pumps, etc.

Thus, if a device is moved from the first floor 100 to the second floor 102, the first locating system 104 will not be able to locate the device on the first floor 102. Due to the range of the wireless antennae or other limitations of the access points 106, 108, and 110, the access points 106, 108, and 110, may not cover the second floor 102. Further, even if such coverage was provided, the triangulation determination would then have to account for three dimensions in order to distinguish between a device located on the second floor 102 versus the first floor 100. Such a calculation may be difficult or impossible for the computation power provided by the location server 122.

To detect if a medical device is moving between floors, such as between the first floor 100 and a second floor 102, the embodiment shown in FIG. 3 may include accessory sensors 136 and 138 near the elevator shaft 132. The embodiment may also include accessory sensors 140 and 142 near the stairwell 134. The accessory sensors may detect if a medical devices entering or leaving a specific floor, thus allowing the location server 122 to determine the location of that medical device based on the access points located on a specific floor.

For example, if the patient 126 and the wearable sensor 128 are initially on the first floor 100, the location server 122 may use the access points 106, 108, and 110 to determine the location of the wearable sensor 128. If the patient 126 decides to leave the first floor 100 using the elevator 130, the patient 126 will first pass by the accessory sensor 136. Similarly, if the patient 126 decides to leave the first floor 100 using the stairwell 134, the patient 126 will pass by the accessory sensor 140. In either case, the accessory sensors 136 or 140 may receive a signal from the active transmitting device of the wearable sensor 128. The accessory sensors 136 or 140 may send a signal to the location server 122, alerting the location server 122 that the wearable sensor 126 has entered the elevator 130 or the stairwell 134. Thus, the location server 122 can store location of the wearable sensor 126 as “in transition.” For a sensor or other medical device in transition, the location server 122 may expect to receive a second signal confirming the destination of the sensor 128 or the medical device. For example, if the patient 126 decides to exit the elevator 130 onto the second floor 102, the patient 126 and the wearable sensor 128 will pass by the accessory sensor 130. Similarly, if the patient 126 is moving between the first floor 100 and the second floor 102 via the stairwell 134, the accessory sensor 142 may send a signal to the location server 122 when the patient 126 exits the stairwell.

As described above, if the location server 122 has stored the status of a medical device, such as the wearable sensor 126, as “in transition,” once the location server 122 receives a signal from an accessory sensor indicating that the medical device has entered a new location, such as the second floor 102, the location server 122 may then change the status of the medical device. Because the location server 122 has verified the new area in which the medical sensor is located, the location server 122 may use the access points located in that area to determine the location of the medical sensor. For example, once the patient 126 and the wearable sensor 128 exit the elevator 130 or the stairwell 134 onto the second floor 102, the location server 122, upon receiving the signal from the accessory sensors 138 or 142, may then use the access points 114, 116, and 118 to determine the specific location of the wearable sensor 128. Thus, by using a combination of access points and accessory sensors, a location system may cover an entire hospital or medical facility. Additionally, the location server 122 may also take specific actions based on data received from the accessory sensors located at the entry and exit points of different areas. For example, in an embodiment discussed above, the location server 122 may provide an alert or notification based on the determined location of a medical device. Similarly, the location server 122 may also provide an alert or notification based on signals received from a specific accessory sensor, such as an accessory sensor leading to a secured area of the hospital or medical facility, leading outside the hospital or medical facility, leading to an employee-only area, etc.

It should be appreciated that any number of locating systems or components thereof may be used in a hospital or other medical facility. For example, multiple location servers could be used on a network or multiple networks, and the location servers may exchange information across a network or multiple networks. The location servers may work in parallel, or each location server may be assigned a different area or different set of access points. In addition, accessory sensors may be used to indicate if a sensor of the medical device is transitioning between locating systems, as opposed to transitioning between areas within a single locating system as described above.

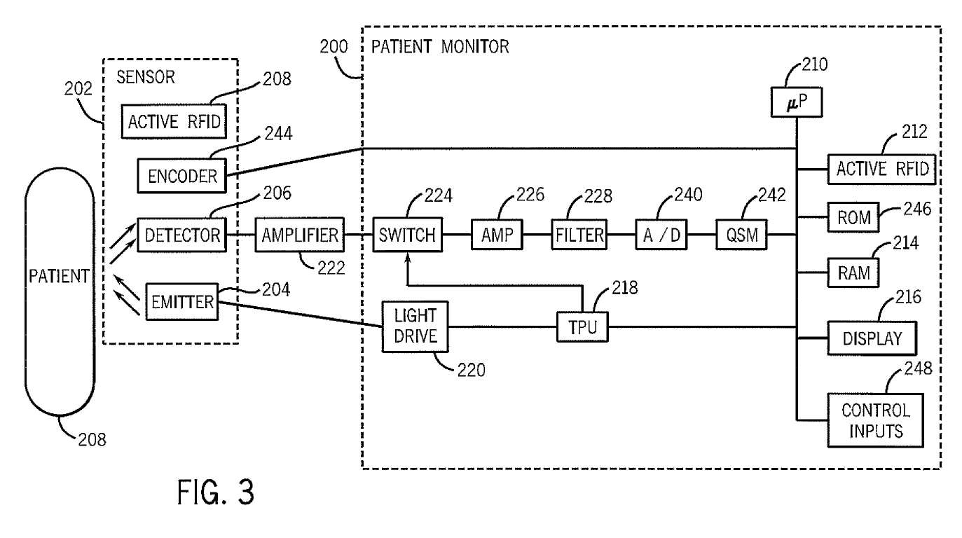

FIG. 4 is a block diagram of one embodiment of a patient monitor 200 and a sensor 202 that may be configured to implement the embodiments of the present disclosure. As described below, the sensor 202 may include an emitter 204 and a detector 206, such as for use with pulse oximetry techniques. However, any sensor capable of reading a physiological parameter may be used with the patient monitor 200 and with the embodiments of the disclosure described. The monitor 202 and sensor 202 may be any suitable monitor and sensor, such as those available from Nellcor Puritan Bennett LLC. To communicate with locating systems described above, the sensor may also include an active RFID device 208.

Turning now to operation of the sensor 202 and the monitor 200, light from emitter 204 passes into the tissue of a patient 208, and is scattered and detected by detector 206. The sensor 202 may be connected to a patient monitor 200. The monitor 200 may include a microprocessor 210 connected to an internal bus 212. Also connected to the bus may be a RAM memory 214 and a display 216. A time processing unit (TPU) 218 may provide timing control signals to light drive circuitry 220 which controls when the emitter 204 is illuminated, and if multiple light sources are used, the multiplexed timing for the different light sources. TPU 220 may also control the gating-in of signals from detector 206 through an amplifier 222 and a switching circuit 224. These signals may be sampled at the proper time, depending upon which of multiple light sources is illuminated, if multiple light sources are used. The received signal from the detector 206 and the contact sensor 202 may be passed through an amplifier 226, a low pass filter 228, and an analog-to-digital converter 240. The digital data may then stored in a queued serial module (QSM) 242 for later downloading to RAM 214 as QSM 242 fills up. In one embodiment, there may be multiple parallel paths of separate amplifier, filter and A/D converters for multiple light wavelengths or spectra received.

A sensor 202 containing an emitter 204 and a detector 206 may also contain an encoder 244 that provides information indicative of the wavelength of light source 220 to allow the monitor to select appropriate calibration coefficients. The encoder 244 may, for instance, be a coded resistor, EEPROM or other coding devices (such as a capacitor, inductor, PROM, RFID, a barcode, parallel resonant circuits, or a colorimetric indicator) that may provide a signal to the processor 210 related to the characteristics of the sensor 202 that may allow the processor 210 to determine the appropriate calibration characteristics for the sensor 202. Further, the encoder 244 may include encryption coding that prevents a disposable part of the sensor 10 from being recognized by a processor 210 that is not able to decode the encryption. Such encryption coding is described in U.S. Pat. No. 6,708,049, which is hereby incorporated by reference in its entirety for all purposes.

Based on the value of the received signals corresponding to the light received by detector 206, the microprocessor 210 may calculate the value of physiological parameter concentration using various algorithms. These algorithms utilize coefficients, which may be empirically determined, corresponding to, for example, the wavelengths of light used. These may be stored in a ROM 246. In a two-wavelength system, the particular set of coefficients chosen for any pair of wavelength spectra may be determined by the value indicated by the encoder 244 corresponding to a particular light source in a particular sensor 10. In one embodiment, multiple resistor values may be assigned to select different sets of coefficients. In another embodiment, the same resistors may be used to select from among the coefficients appropriate for an infrared source paired with either a near red source or far red source. The selection between whether the wavelength sets can be selected with a control input from control inputs 248. Control inputs 248 may be, for instance, a switch on the monitor, a keyboard, or a port providing instructions from a remote host computer. Furthermore, any number of methods or algorithms may be used to determine blood oxygen saturation or any other desired physiological parameter.

In accordance with the embodiments described above, the monitor 200 may also include an active RFID device 250 that may communicate with access points of a locating system as described. Both the active RFID 208 of the sensor 202 and the active RFID 20 of the monitor 200 may be configured to communicate continuously or periodically, such as every 5 minutes, 10 minutes, 15 minutes, 20 minutes, etc. For example, because the sensor 202 may be a wearable sensor, the sensor 202 may be more susceptible to theft or movement to an undesired location. Thus, the active RFID 208 of the sensor 202 may be configured to transmit a signal continuously. Because of the size of the monitor 200 and the relative lack of portability, the active RFID device 250 of the monitor 200 may be configured to transmit a signal periodically.

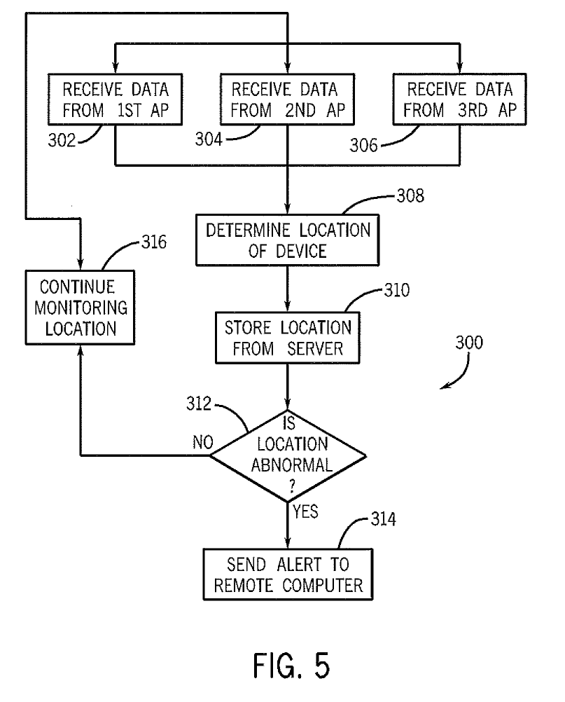

Turning now to FIG. 5, a flowchart depicting a process 300 locating a medical device is shown. As described above, a location server may receive information from multiple access points that correspond to signals received from an active transmitting device on a medical device. The location server and receive data from a first access point (block 302), a second access point (block 304) and a third access point (block 306). Once the location server has received information from at least three access points, the location server may determine the location of the medical device (block 308) via triangulation. The location server may store the determined location of the device (block 310), such as in a database stored on the location server or any other server accessible by the location server via a network.

Once the location of the medical device is determined, the location server may compare the location of the medical device to a list of authorized or unauthorized locations for that specific device, class of devices, and/or patient (decision block 312). If the location of the device is considered abnormal, the location server may send an alert to a remote computer (block 314) which may be monitored by a caregiver, administrator, or any other personnel. Alternatively, the location server may be configured to send an alert to a portable electronic device, such as a cell phone, PDA, etc, or the remote computer may really be alert to such a portable electronic device.

If the determination of the location of the medical device is normal, i.e. if the medical device and/or patient are not located in a non-authorized area, the locating system may continue to monitor the location of the device (block 316). As discussed above, such monitoring may be continuous or periodic, such that the location of the medical device is updated continuously or every 5 minutes, 10 minutes, 15 minutes, 20 minutes etc. Alternatively, in some embodiments a medical device may include an accelerometer or other motion sensing component that can trigger continuous or periodic reporting of the location to the access points if the device is moved.

What is claimed is:

1. A medical device, comprising:
a sensor capable of monitoring a physiological parameter, wherein the sensor is further capable of transmitting location information to one or more wireless communication devices.

2. The medical device of claim 1, wherein the sensor comprises an active transmitting device.

3. The medical device of claim 1, wherein the active transmitting device comprises an active radio frequency identifier.

4. The medical device of claim 1, wherein the sensor is capable of transmitting via a wireless communication protocol.

5. The medical device of claim 1, wherein the sensor comprises a pulse oximetry sensor, an electrocardiogram sensor, a blood glucose sensor, blood pressure sensor, and/or a temperature sensor.

6. The medical device of claim 1, wherein the sensor is capable of sending registration information to one or more wireless communication devices.

7. A medical device, comprising,
a patient monitor configured to monitor a physiological parameter, wherein the monitor is configured to transmit location information to one or more wireless communication devices.

8. The medical device of claim 7, wherein the monitor comprises an active transmitting device.

9. The medical device of claim 7, wherein the monitor's active transmitting device comprises an active radio frequency identifier.

10. The medical device of claim 7, wherein the monitor is configured to actively transmit via a wireless communication protocol.

11. The medical device claim 7, wherein the monitor comprises a pulse oximetry monitor, an electrocardiogram monitor, a blood glucose monitor, a blood pressure monitor, a temperature monitor, or a multi-parameter monitor, or a combination thereof.

12. The medical device of claim 7, wherein the monitor is configured to send registration information.

13. A method of operation, comprising:
receiving a plurality of signals corresponding to a medical device from a plurality of wireless communication devices; and
determining the general location of the medical device from the plurality of signals.

14. The method of claim 13, wherein determining the location of the medical device comprises triangulating the location of the medical device from at least three wireless communication devices.

15. The method of claim 13, comprising storing the location of the medical device.

16. The method of claim 13, comprising comparing the location of the medical device to a list of locations.

17. The method of claim 16, comprising providing a notification and/or an alert based at least in part on the location of the medical device.

18. The method of claim 16, comprising registering the medical device.

19. The method of claim 16, comprising proving the stored locations of the medical device.

20. The method of claim 13, comprising switching between a first plurality of wireless communication devices and a second plurality of wireless communication devices based at least in part on signals received from a monitoring device and/or third plurality of wireless communication devices.
